(19) United States
(12) Patent Application Publication
(10) Pub. No.: US 2025/0332337 A1
(43) Pub. Date: Oct. 30, 2025

(54) DEVICE AND METHOD FOR MONITORING AND DETECTING PATHOGENS

(71) Applicant: Tao Treasures LLC DBA Nanobiofab, Frederick, MD (US)

(72) Inventors: Xiaonao LIU, Frederick, MD (US); Taejun KO, Clarksburg, MD (US); Derese GETNET, Baltimore, MD (US); Deyu LIU, Richland, WA (US); Robert HOPKINS, Frederick, MD (US)

(21) Appl. No.: 19/193,676

(22) Filed: Apr. 29, 2025

Related U.S. Application Data
(60) Provisional application No. 63/640,068, filed on Apr. 29, 2024.

(51) Int. Cl. A61M 1/00; G01N 33/497

(52) U.S. Cl. CPC A61M 1/95; G01N 33/497

(57) ABSTRACT

A device and method are provided for real-time monitoring and detection of pathogen-associated indicators in or derived from medical drainage systems. The device comprises a sensor array configured to detect a plurality of gaseous analytes present in the headspace associated with drainage fluids, wherein said analytes are indicative of microbial presence, metabolic activity, proliferation, or type. An electronic module operatively connected to the sensor array includes an artificial intelligence (AI)-driven algorithm configured to process sensor data, identify a biosignature profile, and generate outputs including pathogen presence, microbial load estimation, pathogen classification, differentiation between infectious and non-infectious inflammation, or prediction of infection risk. The device further includes means for transmitting data and a user interface. The system can be integrated into various drainage setups or function as a standalone unit. The invention also provides methods for continuous or periodic monitoring of drainage fluids.

### CROSS-REFERENCE TO RELATED APPLICATIONS

This application claims the benefit of priority under 35 U.S.C. § 119 to U.S. Provisional Application No. 63/640,068, filed Apr. 29, 2024, the entire content of which is incorporated herein by reference in its entirety.

### TECHNICAL FIELD

The present invention generally relates to a medical device for monitoring and detecting pathogen. More specifically, it relates to such device for monitoring and detecting pathogens in medical drainage systems using a sensor array.

### BACKGROUND

Medical suction drainage systems play a crucial role in the management of surgical and chronic wounds by removing excess fluids from wound sites. Traditional monitoring methods for these drainage systems typically rely on microbiological assays that are not only time-consuming but also delay the detection and treatment of infections, affecting patient outcomes negatively.

Current diagnostic techniques for these systems often lack real-time monitoring capabilities, requiring separate processes to identify pathogens. This results in delays in medical decision-making and effective patient care management. Additionally, existing technologies such as biosensors and chemical assays frequently fall short in achieving the necessary specificity and sensitivity, especially when dealing with complex bodily fluids. These limitations often prevent the detection of all types of pathogens and may require costly and complex setups, which restrict their practical application in a clinical setting.

Moreover, conventional methods may involve invasive procedures to collect sufficient samples, posing additional risks to patients, or rely on indirect signs of infection like changes in fluid temperature or color, which are not always reliable.

On the other hand, current pathogen detection methods, while effective, often rely on slow culture-based techniques that are time-consuming, labor-intensive, and require specialized laboratory facilities. Alternative molecular methods, such as polymerase chain reaction (PCR), offer faster results but still face limitations in accessibility and cost, especially in resource-limited settings.

There is a pressing need for an innovative diagnostic device that can provide real-time, accurate, and non-invasive assessment of microbial status associated with drainage fluids. The proposed invention introduces a sophisticated sensor-based technology capable of detecting a profile of specific gases and volatile compounds present in the headspace above, or dissolved within, the drainage fluid. These analytes, including diverse volatile organic compounds (VOCs), sulfur-containing compounds, nitrogen-containing compounds, and other indicator gases (e.g., CO₂), can be metabolic byproducts or indicators related to the presence, activity, and potentially the type of pathogens, such as bacteria and fungi. This technology allows for near-immediate, direct, and potentially continuous monitoring of indicators associated with infection or microbial proliferation by analyzing the gaseous profile, potentially eliminating the need for time-consuming laboratory culture or complex sample processing.

Designed to be an integral part of various drainage systems, this device utilizes a sensor array coupled with an advanced AI-driven algorithm to enable real-time monitoring, detection, and prediction of infections. This novel approach simplifies operational processes, ensures accurate diagnoses, and significantly enhances infection detection and management in both hospital and home care settings, ultimately improving patient outcomes.

### SUMMARY OF THE INVENTION

The present disclosure relates to a device and method for monitoring and detecting pathogen. The system contains a sensor device configured to detect pathogens by analyzing gaseous emissions from microorganisms that are present in medical drainage systems.

The system provides real-time monitoring of the metabolically active pathogens via their produced gaseous chemical evidence without the need for invasive sample processing or wound exposure.

Accordingly, one aspect of the present invention relates to a device and system for the analysis of drainage fluids to provide near real-time, accurate, and potentially non-invasive assessment of microbial status and infection. Addressing the limitations of traditional diagnostic methods which often involve delays and complex sample processing, the invention utilizes advanced sensor technology coupled with intelligent data analysis. The device typically comprises:
a) a sensor array configured to detect a plurality of analytes, primarily gaseous indicators present in the headspace above or dissolved within the drainage fluid. Such analytes include, but are not limited to, various volatile organic compounds (VOCs), sulfur-containing compounds, nitrogen-containing compounds, and other indicator gases (e.g., CO₂) indicative of the presence, metabolic activity, proliferation, or type of microorganisms (e.g., bacteria, fungi), and potentially indicators of host inflammatory response. The array may optionally include sensors for physical or other chemical properties (e.g., color, pH, temperature);
b) an intelligent analysis module, such as an AI-driven algorithm, operatively connected to the sensor array. This module is configured to process the multi-sensor data streams, perform feature extraction, recognize characteristic patterns or signatures (i.e., a “biosignature profile”), and apply computational models (e.g., machine learning algorithms trained on data correlating sensor patterns with known clinical and microbiological outcomes, including bacterial load data) to interpret these patterns. The interpretation aims to generate outputs such as: an indication of pathogen presence (e.g., positive/negative screening for infection), providing an estimation of microbial load correlated with bacterial concentration (e.g., expressed in estimated Colony Forming Units per milliliter or CFU/mL), an assessment aiding in pathogen identification or classification (e.g., discriminating between bacterial types or identifying fungi), differentiation between infectious and non-infectious inflammatory states, and/or prediction of infection risk;
c) communication means, potentially including wireless connectivity options, for transmitting sensor data and analysis results for remote monitoring, data logging, or further analysis;
d) optionally, a user interface comprising display means (e.g., LED or LCD screen) and alert systems (e.g., visual or audible alarms) to present results, status, or warnings; and
e) a versatile physical configuration allowing for integration into or association with existing drainage systems (e.g., in-line with drainage tubing, connected to a collection reservoir, or incorporated into a wound dressing assembly) or for use as a standalone unit analyzing collected samples.

The outputs generated by the device can serve various clinical and laboratory purposes, such as providing an early indication of infection, facilitating rapid triage of samples or patients, informing acute clinical decisions potentially including initial antibiotic guidance, and optimizing laboratory workflow efficiency.

The sensor array comprises electrochemical sensors, infrared sensors, photoionization detectors, humidity sensors, thermal sensors, pH sensors, viscosity sensors, and chemiresistive sensors made from materials including conductive polymers, carbon nanotubes, graphene, transition metal chalcogenides, metal nitrides, metal sulfides, MXenes, metal-organic frameworks (MOFs), composite nanomaterials, and various metal oxides.

The sensor array is highly sensitive and capable of detecting pathogen concentrations across a broad range, from 1 to 10⁸ colony-forming units (CFU) per mL of fluid. This sensitivity is crucial for early-stage infection detection, allowing for immediate clinical response before traditional culture-based methods would yield results.

This versatility is achieved through several potential configurations. For example, the device may be configured for in-line monitoring, wherein the sensor array is placed directly within the flow path of the drainage tubing to continuously analyze the fluid as it passes. Alternatively, the device may be connected to an existing drainage system component, such as the collection reservoir or tubing, allowing it to sample headspace gases (e.g., VOCs) from a portion of the fluid. In another embodiment, the sensor components could be integrated into the structure of the drainage tube or catheter itself. Furthermore, the device is designed to function effectively as a standalone unit, allowing for the analysis of drainage fluid samples that have been collected separately.

As described, central to this invented device is its ability to integrate into existing medical suction drainage systems—such as Jackson-Pratt, Hemovac, Blake, Redon, Penrose, Vacuum-Assisted Closure (VAC), Capillary, Silicone drains, and other types—or operate as a standalone unit. This versatility allows for broad application across various medical settings, from hospitals to outpatient clinics.

Utilizing an AI-driven algorithm, the device processes data from the sensor array to provide real-time monitoring and detection of pathogens, and prediction of infections. This enables healthcare providers to swiftly identify the presence, type, and growth rates of pathogens, facilitating timely and effective medical interventions.

The device offers wireless connectivity options that support remote monitoring capabilities. This feature enables healthcare providers to receive real-time data and make informed decisions about treatment plans from afar, thus improving the management of infections and reducing the risk of severe complications.

The incorporation of the headspace gas detection technology into the device allows for the continuous monitoring of the gas composition over time, providing dynamic insights into the progression or resolution of an infection. The data collected from the headspace offers a non-invasive, rapid-response method to preemptively alert medical staff to changes in the patient's condition that may necessitate intervention, well before visible signs of infection become apparent.

In one embodiment, beyond detecting gases, this device is further configured to analyze the color and volume of drainage fluids with optical sensing technology, providing clinicians with essential insights to track medical conditions more effectively. This capability enables a comprehensive assessment of the drainage fluid's characteristics, which are critical for ongoing clinical evaluations and timely interventions.

In another embodiment, the device includes an integrated digital screen that directly displays results and information. In still another embodiment, the device provides visual and audible alerts to notify healthcare providers of critical changes or potential issues, enhancing the immediacy and effectiveness of medical responses.

Another aspect of the invention relates to a method for monitoring and detecting pathogenic infections in a medical drainage system. The method comprising:
a) applying the above-described device to detect at least one airborne compound emitted from the drainage fluid of the medical drainage system;
b) processing data from the sensor array using the AI-driven algorithm for real-time monitoring, detection, and prediction of pathogenic infections;
c) utilizing wireless connectivity options for remote monitoring and data analysis;
d) displaying diagnostic results and providing visual and audible alerts through the user interface.

Yet another aspect of the invention relates to a medical drainage system integrated with the above-described device, wherein the device can detect specific metabolites/gas (like VOC) profiles indicative of pathogenic infections from drainage fluids.

Examples of the medical drainage system can be selected, but not limited to, Jackson-Pratt, Hemovac, Blake, Redon, Penrose, specifically Vacuum-Assisted Closure (VAC)/Negative Pressure Wound Therapy (NPWT) systems, Capillary drains, Chest Tubes, Pigtail drains, and Silicone drains.

With the medial drainage system, the integrated device includes a sensor array for continuous scanning of at least one specific metabolite sensing profile that indicates infection.

Another aspect of the invention relates to a method for enriching and detecting metabolites produced by pathogens in drainage fluids using the as-introduced medical drainage system. Advantages of the method include no need for sample processing.

The details of the invention are set forth in the drawing and the description below. Other features, objects, and advantages of the invention will be apparent to those persons skilled in the art upon reading the drawing and the description, as well as from the appended claims.

### BRIEF DESCRIPTION OF THE DRAWINGS

FIG. 1 is a schematic illustration of a medical drainage system comprising a sensor device positioned in-line with drainage tubing between a patient and a drainage reservoir. The sensor device is configured to capture gas-phase analytes emitted from wound exudate without direct liquid contact.

FIG. 2 is a cross-sectional diagram of the sensor device showing multiple sensing modalities interfacing with the drainage fluid. Gas-phase sensors (e.g., chemiresistive sensor, optical sensor) are positioned above the fluid path, while liquid-contact sensors (e.g., pH sensor, viscosity sensors, temperature sensor, biosensor) are positioned below.

FIGS. 3A-3D illustrates various sensor device configurations for pathogen monitoring:

FIG. 3A: Inline—sensor device integrated into the drainage tubing;

FIG. 3B: Attached—sensor device attached externally to the drainage reservoir;

FIG. 3C: Integrated—sensor device built into the drainage reservoir for continuous monitoring;

FIG. 3D: Standalone—analyzer device configured to receive discrete aspirated samples from the wound or reservoir.

### DETAILED DESCRIPTION

The following detailed description, in conjunction with the accompanying drawings, provides a more complete understanding of the disclosure and its various embodiments. The description is not intended to be limiting, and modifications and variations within the scope of the disclosure will be apparent to those skilled in the art.

The present invention encompasses a medical diagnostic device specifically engineered for real-time, accurate, and non-invasive detection of infections in suction drainage systems. This innovation is primarily aimed at enhancing patient care by providing swift diagnostic capabilities directly at the point of care. The device includes a sensor array, data processing unit, and user interface.

The various embodiments of the device and method include, but not limited to, the embodiments as disclosed, as disclosed, described, and/or referred to in the following applications: U.S. application Ser. No. 18/829,748, filed Sep. 10, 2024; and U.S. application Ser. No. 18/933,674, filed Oct. 31, 2024, which are all hereby incorporated in reference by their entireties. The embodiments in these applications herein incorporated can be regarded in combination with one another or as a single invention, rather than as discrete and independent filings.

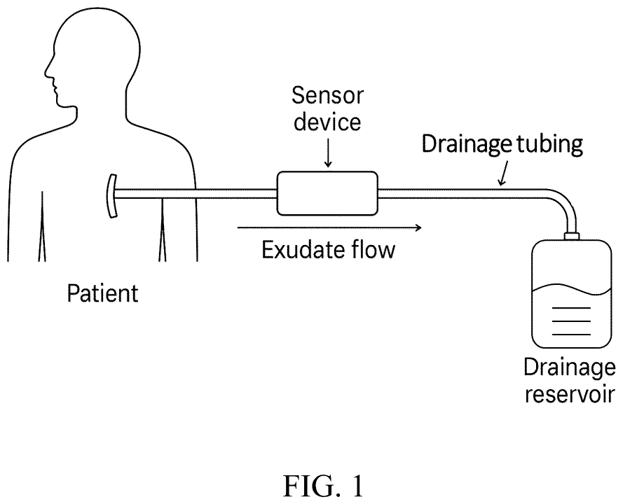

FIG. 1 illustrates a wound drainage system comprising a patient, drainage tubing, a sensor device, and a drainage reservoir. Wound exudate flows from the patient through the drainage tubing toward the drainage reservoir. The sensor device is positioned inline along the drainage tubing (2), preferably close to the reservoir, and is configured to detect volatile organic compounds (VOCs) and other gas-phase analytes emitted from the exudate without requiring direct contact with the liquid.

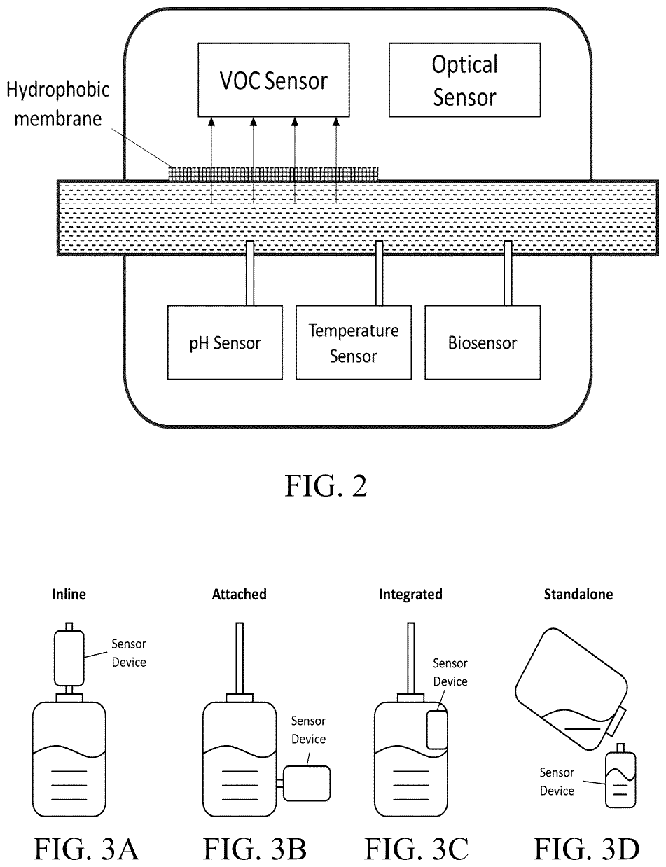

FIG. 2 shows a cross-sectional schematic of the sensor device. The sensor device includes a fluid channel through which wound exudate flows. Above the fluid channel, a gas-permeable, liquid-impermeable membrane permits VOCs and other gaseous analytes to diffuse into a gas-phase sensor array, including gas sensors and optionally an optical sensor. Below the fluid channel, liquid-contact sensors such as a pH sensor, viscosity sensors, and a temperature sensor are positioned to monitor properties of the exudate.

FIGS. 3A-3D depicts alternative sensor device configurations for use with medical drainage systems:

FIG. 3A (Inline) shows the sensor device integrated into the drainage tubing upstream of the drainage reservoir, configured to monitor gas emissions in real time during fluid transport.

FIG. 3B (Attached) shows the sensor device externally attached to the drainage reservoir, allowing gases to be monitored from the reservoir's headspace without disturbing fluid flow.

FIG. 3C (Integrated) shows a sensor device embedded directly into the structure of the drainage reservoir, forming an integrated monitoring unit that enables continuous assessment of wound exudate emissions.

FIG. 3D (Standalone) shows a standalone analyzer system, configured to receive aspirated wound fluid samples from the drainage system or wound site for offline analysis using the integrated sensor array.

The core component of the device is an advanced sensor array equipped to detect volatile organic compounds (VOCs) and other gases that are indicative of pathogenic activity in drainage fluids such as blood, pus, serous fluid, serosanguineous fluid, seropurulent fluid, lymph, bile, intestinal contents, and other bodily secretions.

The sensor array comprised of multiple sensors capable of detecting metabolites, volatile organic compounds (VOCs), pathogens, and fluid properties. It can detect and analyze the biochemical markers indicative of bacterial, fungal, or viral infections and fluid characteristics directly in the drainage system or from a collected sample.

The sensor array uses one, or two, or more sensors, or a combination of thereof, selected from the group consisting of, for example, chemiresistive sensors (e.g., metal oxide semiconductor (MOS) sensors), optical (utilizing visible and/or near infrared wavelengths to detect changes in optical properties such as color, turbidity, absorbance, reflectance, or fluorescence) sensors, humidity sensors, electrochemical sensors, pH sensors, viscosity sensors, temperature sensors, pressure sensors, impedance/conductometric sensors, and/or specific biosensors (e.g., antibody-, enzyme-, or aptamer-based sensors), to detect physical or chemical changes in the drainage fluid or headspace gases caused by or indicative of the metabolism of pathogens. Such changes may include, but are not limited to, the production or consumption of gases, shifts in pH or temperature, changes in fluid color, optical density or turbidity, alterations in electrical impedance or conductivity, variations in humidity, or the presence of specific biological markers or metabolites. Employing an array or combination of different sensor types can enhance detection specificity, sensitivity, robustness against interfering factors, and provide a more comprehensive assessment of pathogenic activity by monitoring multiple indicators simultaneously.

Designed for versatility, the device can be integrated operatively connected into or associated with various existing medical drainage or suction systems, for example, as an inline component within the drainage tubing, connected to a port on the collection reservoir, or incorporated directly into a wound dressing assembly. Such systems include products from, but are not limited to, Jackson-Pratt, Hemovac, Blake, Redon, Penrose, specifically Vacuum-Assisted Closure (VAC)/Negative Pressure Wound Therapy (NPWT) systems, Capillary drains, Chest Tubes, Pigtail drains, and Silicone drains. Alternatively, or additionally, it can function effectively as a standalone unit, for instance, by receiving collected fluid samples introduced via a sample port, cartridge, or into a dedicated analysis chamber, making it suitable for a wide range of medical environments from large hospitals to field settings, home care environments, or smaller outpatient clinics.

In one or more embodiment, the chemiresistive sensors include one or more sensing materials such as, but not limited to, conductive polymers (e.g., polyaniline (PANI), polypyrrole (PPy), PEDOT:PSS), carbon nanotubes (CNTs, e.g., single-walled or multi-walled), graphene or graphene oxide, other two-dimensional (2D) materials like transition metal dichalcogenides (TMDs, e.g., molybdenum disulfide (MoS2), WS2), metal sulfides, conductive metal-organic frameworks (MOFs), metal nitrides, and/or various metal oxides (e.g., zinc oxide, tin oxides, tungsten oxides, titanium oxides, indium oxide). These materials may be configured, for example, as films, nanostructures, composites, or coatings on additional substrate materials. The sensors comprising these materials change their electrical resistance, capacitance, or other electronic properties in response to interactions with target analytes such emitted or consumed by pathogens present in the drainage fluid's headspace, such as ammonia, hydrogen sulfide, indole, amines, alcohols, ketones, volatile fatty acids, or other metabolic byproducts. Depending on the specific sensing material and configuration, the sensors may operate at room temperature or may require thermal activation or heating to achieve optimal sensitivity and selectivity. Further, the sensor material comprises unary, binary, ternary, quaternary, quinary, senary, septenary, and octonary multiple-component metal oxides.

The sensing materials employed in the sensor array may comprise one or more elements, or compounds thereof (e.g., oxides, sulfides, nitrides, conductive polymers, composite materials). Suitable elements that may form part of the sensing material, particularly for metal oxide, metal sulfide, or metal nitride sensors, include, but are not limited to: Sn, Co, Zn, In, Cu, Ni, Cr, Mn, W, Ti, V, Fe, Al, Ga, Ag, Au, Pd, Pt, Si, Ce, Mo, Zr, La, Y, Mg, Nb, Ru, and Te. These elements can be used individually (e.g., in elemental form for certain electrochemical applications) or, more commonly, combined to form compounds suitable for sensing applications. For example, the sensing material may comprise binary, ternary, or more complex compositions. Illustrative examples of suitable sensing materials include, but are not limited to:
A) Binary Metal Oxides: Pure or mixed phases of binary composition, such as Tin Oxide (e.g., SnO₂), Zinc Oxide (e.g., ZnO), Tungsten Oxide (e.g., WO₃), Titanium Dioxide (e.g., TiO₂), Copper Oxide (e.g., CuO, Cu₂O), Nickel Oxide (e.g., NiO), Iron Oxide (e.g., Fe₂O₃, Fe₃O₄), Indium Oxide (e.g., In₂O₃), Cerium Oxide (e.g., CeO₂), Zirconium Oxide (e.g., ZrO₂), Manganese Oxide (e.g., MnO₂, Mn₃O₄), Cobalt Oxide (e.g., CoO, Co₃O₄), Gallium Oxide (e.g., Ga₂O₃), Molybdenum Oxide (e.g., MoO₃), Magnesium Oxide (e.g., MgO), or Niobium Oxide (e.g., Nb₂O₅).
B) Mixed or Ternary Metal Oxides: May be consists by pure ternary crystal phase or doped binary phases, such as Zinc Stannate (e.g., Zn₂SnO₄), Indium Tin Oxide (ITO), Aluminum-doped Zinc Oxide (AZO), Fluorine-doped Tin Oxide (FTO), perovskite oxides (e.g., SrTiO₃, LaCoO₃, BaTiO₃), or spinel oxides (e.g., NiFe₂O₄, NiCo₂O₄, ZnFe₂O₄).
C) Metal Sulfides or Nitrides: Such as Molybdenum Disulfide (MoS₂), Tungsten Disulfide (WS₂), Tin Sulfide (e.g., SnS, SnS₂), Indium Sulfide (e.g., In₂S₃), Gallium Nitride (GaN), Aluminum Nitride (AlN), Indium Nitride (InN), or Titanium Nitride (TiN).
D) Noble Metal Catalysts/Additives: Noble metals like Au, Pt, Pd, Ag, Ru, Rh may be included, for example, as dopants or catalytic site loadings incorporated into the crystal lattice, or as nanoparticles decorating the surface of the primary sensing material, or as layers to enhance chemical kinetics to improve sensitivity, selectivity, or response/recovery times (e.g., Pd-doped SnO₂, Au nanoparticles on ZnO, Pt-functionalized WO₃).
E) Carbon-Based Materials: Diverse form of elemental carbon or other carbonaceous structures, such as graphene, graphene oxide, reduced graphene oxide (rGO), carbon nanotubes (CNTs), which may be used alone or functionalized or composited with other materials (e.g., metal oxides, polymers).
F) Conductive Polymers: Such as polyaniline (PANI), polypyrrole (PPy), PEDOT:PSS, which may be used alone or in composites.
G) Combinations: Direct mixtures, composites (e.g., polymer-metal oxide composites, CNT-metal oxide composites), or heterostructures (e.g., p-n junctions like CuO/ZnO) comprising two or more of the above materials or material types.

Where formulas like MₓO_{y}, MₓM_{y}′O_{z}, etc., are used herein, the subscript “x,y,z” indicates that the compound may encompass variable stoichiometry, non-stoichiometry, or different oxidation states of the constituent elements, as is common for many materials, particularly metal oxides, sulfides, and nitrides, used in sensing applications. These materials are selected based on their ability to exhibit measurable changes in their electrical (e.g., resistance, conductance, capacitance, work function), optical (e.g., absorbance, reflectance, fluorescence, color), chemical (e.g., reactivity), or physical (e.g., mass, temperature) properties upon interaction with target analytes (e.g., gases, VOCs, ions) present in the drainage fluid or associated headspace gas.

The electrochemical sensors that may be included in the sensor array comprise one or more types, including, but not limited to, amperometric sensors (which measure current changes due to redox reactions at a set potential, e.g., related to concentrations of dissolved oxygen, hydrogen peroxide, glucose, lactate, or specific redox-active metabolites or virulence factors), potentiometric sensors (which assess potential differences between electrodes, e.g., using ion-selective electrodes (ISEs) to measure pH or specific ion concentrations like K⁺, Na⁺, Cl⁻, Ca²⁺), conductometric or impedimetric sensors (which measure changes in the electrical conductivity or impedance of the fluid across a range of frequencies, potentially related to overall ionic strength, salinity, cell lysis, or bacterial concentration/biofilm formation), and/or voltametric sensors (e.g., cyclic voltammetry, differential pulse voltammetry, which measure current as potential is varied to characterize redox species). These sensors typically utilize a configuration of three electrodes, including working, reference (e.g., Ag/AgCl), and counter electrodes, which may be fabricated from but not limited to platinum, gold, carbon (e.g., glassy carbon, screen-printed carbon), conductive polymers, tailored to detect specific analytes or changes in the electrochemical properties of the drainage fluid.

Humidity sensors are integrated into the array to monitor the moisture content of the drainage fluid. These sensors provide critical data for assessing the state of the fluid and identifying deviations that might suggest pathogenic activities or other changes in the wound environment, as well as support the calibration of the work condition of other sensors.

Optical sensors are employed to monitor the amount and analyze the optical properties of fluids, such as turbidity, color, and fluorescence. These sensors operate across different ranges, including visible light and near-infrared. These sensors are calibrated to detect specific pathogenic markers that become visually apparent in the fluid, aiding in the rapid identification of infection.

Thermal sensors within the sensor array provide real-time monitoring of the fluid and environment temperature. The thermal sensors are capable of detecting minute temperature variations in the fluid, which can be indicative of metabolic heat produced by growing pathogens or inflammatory responses from the host.

pH sensors are included to measure the pH of the drainage fluids. Signal fluctuations can be indicative of infection or other biochemical changes at the drainage site, providing essential diagnostic information.

Utilizing an AI-driven algorithm, the device processes data from the sensor array to provide real-time monitoring, detection, and predictive analysis of pathogenic infections. This AI algorithm is to rapidly assess the presence, type, and growth rates of pathogens, thereby facilitating timely and effective medical interventions.

The advanced algorithms include but not limited to machine learning, deep learning, neural networks, and predictive analytics, to detect and predict infections based on real-time data inputs. These AI techniques may involve supervised learning, unsupervised learning, reinforcement learning, and other data-driven methodologies that enhance the system's ability to identify patterns indicative of pathogen presence and infection risk. By analyzing a continuous stream of sensor data, the algorithms provide precise, timely insights that assist clinicians in making informed decisions regarding infection management.

The device has a sensor array that responds to more or more compounds in the gas phase, including but not limited to VOCs, sulfur-containing compounds, and nitrogen-containing compounds (e.g. ammonia), carbon dioxide (CO₂). These molecules are metabolic byproducts of pathogens such as bacteria and fungi, and their presence in the wound environment is indicative of microbial activity.

The device has a sensor array configured to respond to one or more compounds in the gas phase (e.g., within the headspace above the drainage fluid or wound site), including but not limited to: VOCs (such as short-chain acids (e.g., acetic acid, propionic acid, butyric acid, isovaleric acid), alcohols (e.g., ethanol, isopropanol), aldehydes, ketones (e.g., acetone), esters, amines, and terpenes); sulfur-containing compounds (e.g., hydrogen sulfide, dimethyl sulfide, dimethyl disulfide, methanethiol); and nitrogen-containing compounds (e.g., ammonia, trimethylamine, indole, skatole); as well as other indicator gases like CO₂. These molecules can be metabolic byproducts or indicators of pathogens such as bacteria (e.g., Staphylococcus aureus, Pseudomonas aeruginosa, Escherichia coli, Proteus mirabilis, anaerobes like Bacteroides spp. or Clostridium spp.) and fungi (e.g., Candida species), and their presence, concentration, relative abundance, or specific temporal pattern in the headspace or dissolved in the wound environment/drainage fluid is indicative of microbial activity, proliferation, virulence, or specific metabolic pathways often associated with infection. The detected profile of these compounds may help in assessing the microbial load or even discriminating between different types of pathogens or between infection and non-infected inflammation states (whereby some VOCs may also originate from the host inflammatory response).

The sensor array may include one or more types of gas sensors, including but not limited to resistive, capacitive, electrochemical, optical, impedance, photoionization, field-effect transistor (FET) sensors, and surface plasmon resonance (SPR) sensing module, allowing detection of one or more chemical compounds.

The resistive gas sensors are chemiresistive electronics made from a range of materials, including but not limited to metal oxide, carbon-based, polymers, nanomaterials, composite materials, silicon-based materials, noble metals, and transition metal chalcogenide materials. Each material provides unique responses answering to their properties. The sensors are screened based on criteria such as enhanced sensitivity, selectivity, stability or robustness, tailored for detecting gases emitted by pathogenic organisms. The sensor is calibrated to detect gases at concentrations ranging from 1 ppb to 1000 ppm, typically emitted by pathogenic organisms, which may indicate infection or colonization in a wound environment.

The sensor array displays exceptional sensitivity, capable of detecting pathogen concentrations ranging from 1 to 10⁸ CFU per mL of fluid. This high level of sensitivity is critical for the early detection of infections, allowing healthcare providers to respond immediately, well before results from traditional culture-based methods are available.

The device includes wireless connectivity options such as Bluetooth, WiFi, and Near Field Communication (NFC), which support extensive remote monitoring capabilities. These features enable healthcare providers to receive and analyze real-time data remotely, making informed decisions about treatment plans, thus improving the management of infections and reducing the risk of severe complications.

Beyond its ability to detect gases, the device is specifically designed to analyze physical characteristics of the drainage fluids such as color and volume. The sensor array uses one, or two, or more, or a combination of aforementioned types of sensors, including optical sensors, electrochemical sensors, pH sensors, viscosity sensors, humidity sensors, temperature sensors, and chemiresistive sensors to perform multi modal detection of characteristic changes in high dimensional data space. This analysis provides clinicians with vital information necessary for tracking the progression of medical conditions and adjusting treatment plans accordingly.

An integrated screen on the device displays diagnostic results and other relevant information directly to the user. Additionally, the device is equipped with systems that provide both visual and audible alerts to notify healthcare providers of critical changes in the patient's condition or potential issues with the device's operation.

The sensor device is configured to operate in real-time, enabling continuous monitoring of fluid characteristics and pathogen-related markers directly within the drainage system, as illustrated in FIGS. 3A-3C. In some embodiments, the sensor device may alternatively be used to analyze collected fluids after drainage, as illustrated in FIG. 3D, providing immediate diagnostic assessment of patient condition without the need for external laboratory processing.

The device offers operational flexibility to accommodate different clinical workflows. In certain embodiments, the sensor device is positioned in-line with the drainage tubing (FIG. 3A), attached externally to the reservoir (FIG. 3B), or integrated into the reservoir structure (FIG. 3C), allowing real-time monitoring during fluid transport. In another embodiment, the device functions as a standalone analyzer configured to assess fluid samples collected separately (FIG. 3D), enabling rapid point-of-care diagnostics.

Designed for easy integration into any existing suction drainage system or as a standalone unit adaptable to various medical environments. Offers flexibility in deployment, making it suitable for use in hospitals, clinics, and field operations, enhancing the range of applications in patient care.

The pathogens of wound infection can be one or more bacterial or fungi species, such as Acinetobacter anitratus, Acinetobacter baumannii, Actinomyces israelii, Agrobacterium radiobacter, Agrobacterium tumefaciens, Anaplasma phagocytophilum, Aspergillus fumigatus, Azorhizobium caulinodans, Azotobacter vinelandii, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacillus thuringiensis, Bacteroides fragilis, Bacteroides gingivalis, Bacteroides melaninogenicus, Bartonella henselae, Bartonella quintana, Bordetella bronchiseptica, Bordetella pertussis, Borrelia burgdorferi, Brucella abortus, Brucella melitensis, Brucella suis, Burkholderia mallei, Burkholderia pseudomallei, Burkholderia cepacia, Calymmatobacterium granulomatis, Campylobacter coli, Campylobacter fetus, Campylobacter jejuni, Candida albicans, Helicobacter pylori, Chlamydia trachomatis, Chlamydophila pneumoniae, Chlamydophila psittaci, Clostridium botulinum, Clostridium difficile, Clostridium perfringens, Clostridium tetani, Corynebacterium diphtheriae, Candida species, Corynebacterium fusiforme, Coxiella burnetii, Ehrlichia chaffeensis, Enterobacter cloacae, Enterococcus avium, Enterococcus durans, Enterococcus faecalis, Enterococcus faecium, Enterococcus gallinarum, Enterococcus maloratus, Escherichia coli, Francisella tularensis, Fusobacterium nucleatum, Enterobacter species, Fusarium solani, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus acidophilus, Lactobacillus bulgaricus, Lactobacillus casei, Lactobacillus delbrueckii, Lactococcus lactis, Legionella pneumophila, Listeria monocytogenes, Methylobacterium extorquens, Microbacterium multiforme, Micrococcus luteus, Moraxella catarrhalis, Morganella morganii, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Mycoplasma mexicoense, Neisseria gonorrhoeae, Neisseria meningitidis, Pasteurella multocida, Pasteurella tularensis, Porphyromonas gingivalis, Prevotella melaninogenica, Proteus vulgaris, Proteus mirabilis, Proteus penneri, Providencia stuartii, Pseudomonas aeruginosa, Rhizobium radiobacter, Rickettsia prowazekii, Rickettsia psittaci, Rickettsia quintana, Rickettsia rickettsii, Rickettsia trachomae, Rochalimaea henselae, Rochalimaea quintana, Rothia dentocariosa, Salmonella enteritidis, Salmonella typhi, Salmonella typhimurium, Serratia marcescens, Shigella dysenteriae, Spirillum volutans, Staphylococcus aureus, Staphylococcus epidermidis, Stenotrophomonas maltophilia, Streptococcus agalactiae, Streptococcus avium, Streptococcus bovis, Streptococcus cricetus, Streptococcus faecium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema pallidum, Treponema denticola, Vibrio cholerae, Vibrio comma, Vibrio parahaemolyticus, Vibrio vulnificus, Yersinia enterocolitica, Yersinia pestis and Yersinia pseudotuberculosis, and/or known to include one or more antibiotic-resistant strains descending from a known species, and/or known to comprise one or more extended spectrum beta-lactamase-producing strains descending from a known species, in particular the one or more extended spectrum beta-lactamase-producing strain is selected from the group consisting of: extended spectrum beta-lactamase-producing Escherichia coli, and extended spectrum beta-lactamase-producing Klebsiella pneumoniae. The fungal species include but not limited to Aspergillus Species, Candida Species, Fusarium Species, Mucorales (Zygomycetes), Scedosporium Species, Curvularia Species, Alternaria Species, Trichophyton Species, Exophiala Species, Cladosporium Species, Bipolaris Species, Penicillium Species, Phialophora and Fonsecaea Species (Agents of Chromoblastomycosis).

The Antibiotic-resistant bacterial strains may include Carbapenem-resistant Acinetobacter baumannii, carbapenem-resistant Pseudomonas aeruginosa, vancomycin-resistant Enterococcus faecium, methicillin-resistant Staphylococcus aureus, vancomycin-resistant Staphylococcus aureus, clarithromycin-resistant Helicobacter pylori, fluoroquinolone-resistant Campylobacter coli, fluoroquinolone-resistant Campylobacter fetus, fluoroquinolone-resistant Campylobacter jejuni, fluoroquinolone-resistant Helicobacter pylori, fluoroquinolone-resistant Salmonella enteritidis, fluoroquinolone-resistant Salmonella typhi, fluoroquinolone-resistant Salmonella typhimurium, cephalosporin-resistant Neisseria gonorrhoeae, fluoroquinolone-resistant Neisseria gonorrhoeae, penicillin-non-susceptible Streptococcus pneumoniae, ampicillin-resistant Haemophilus influenzae, fluoroquinolone-resistant Shigella dysenteriae, carbapenem-resistant Escherichia coli, carbapenem-resistant Klebsiella pneumoniae, carbapenem-resistant Enterobacter cloacae, carbapenem-resistant Serratia marcescens, carbapenem-resistant Proteus vulgaris, carbapenem-resistant Proteus mirabilis, carbapenem-resistant Proteus penneri, carbapenem-resistant Providencia stuartii, carbapenem-resistant Morganella morganii, cephalosporin-resistant Escherichia coli, cephalosporin-resistant Klebsiella pneumoniae, cephalosporin-resistant Enterobacter cloacae, cephalosporin-resistant Serratia marcescens, cephalosporin-resistant Proteus vulgaris, cephalosporin-resistant Proteus mirabilis, cephalosporin-resistant Proteus penneri, cephalosporin-resistant Providencia stuartii and cephalosporin-resistant Morganella morganii.

After an infection has been identified and treated, the device continues to monitor gas emissions for pathogen fingerprints. The sensor device can be reset to tare mode for recalibration if necessary, allowing for continuous surveillance of pathogen.

While the present disclosure has been described with reference to specific embodiments, it will be understood by those skilled in the art that various changes may be made, and equivalents may be substituted without departing from the scope of the disclosure. In addition, many modifications may be made to adapt a particular situation or material to the teachings of the disclosure without departing from the scope thereof. Therefore, it is intended that the present disclosure is not limited to the particular embodiments disclosed, but that the disclosure will include all embodiments falling within the scope of the appended claims.

We claim:

1. A device for monitoring and detecting a pathogen-associated indicator in or derived from a medical drainage system, comprising:
(a) a sensor array configured to detect a plurality of gaseous analytes present in a headspace associated with drainage fluid medical drainage system, wherein said gaseous analytes are indicative of microbial presence, metabolic activity, proliferation, or type, and comprise one or more compounds selected from the group consisting of volatile organic compounds, alcohols, aldehydes, ketones, esters, terpenes, sulfur-containing compounds, nitrogen-containing compounds, and CO₂;
(b) an electronic module operatively connected to the sensor array, said module comprising an artificial intelligence (AI)-driven algorithm configured to: i) process data received from the sensor array; ii) identify a characteristic biosignature profile based on the processed data; and iii) generate an output based on interpretation of the biosignature profile, said output comprising at least one of the followings: an indication of pathogen presence, an estimation of microbial load, an assessment aiding in pathogen classification or identification, a differentiation between infectious and non-infectious inflammatory states, or a prediction of infection risk;
(c) communication means for transmitting data or the generated output; and
(d) a user interface configured to display outputs or provide an alert based thereon.

2. The device of claim 1, wherein the sensor array comprises one or more sensors selected from the group consisting of: chemiresistive sensors, electrochemical sensors, optical sensors, infrared sensors, photoionization detectors, humidity sensors, thermal sensors, pH sensors, and viscosity sensors.

3. The device of claim 2, wherein the chemiresistive sensors comprise a sensing material selected from the group consisting of: metal oxides, conductive polymers, carbon materials, metal chalcogenides, metal nitrides, MXenes, metal-organic frameworks, composite nanomaterials.

4. The device of claim 2, wherein the electrochemical sensors are selected from the group consisting of: amperometric sensors, potentiometric sensors, conductometric sensors, impedimetric sensors, and voltametric sensors, optionally comprising working, reference, and counter electrodes.

5. The device of claim 1, further comprising one or more liquid-contact sensors configured to measure a property of the drainage fluid selected from the group consisting of: pH, temperature, viscosity, color, turbidity, optical density, impedance, and conductivity.

6. The device of claim 1, wherein the sensor array and intelligent analysis module are configured to provide an estimation of microbial load correlated with pathogen concentrations ranging from 1 to 10⁸ colony-forming units (CFU) per milliliter of fluid.

7. The device of claim 1, wherein the AI-driven algorithm employs a machine learning or deep learning model trained on data correlating sensor array patterns with known clinical and microbiological outcomes, including specific pathogen identities or microbial loads.

8. The device of claim 1, wherein the AI-driven algorithm is further configured for self-learning from accumulated sensor data over time to improve accuracy or predictive capability.

9. The device of claim 1, wherein the communication means comprises wireless connectivity supporting Bluetooth, Wi-Fi, or Near Field Communication (NFC), enabling remote monitoring.

10. The device of claim 1, wherein the user interface comprises a digital display screen and provides visual or audible alerts for critical diagnostic results or device status.

11. The device of claim 1, wherein the device is configured for operation in a mode selected from the group consisting of: in-line within drainage tubing, attached to a drainage system component, integrated into a drainage reservoir, integrated into a wound dressing, and as a standalone unit analyzing a collected sample, optionally in a laboratory or field setting.

12. The device of claim 1, wherein the pathogens detectable include bacterial species and fungal species commonly associated with surgical or chronic wound infections.

13. The device of claim 12, wherein:
a) the pathogen comes from bacterial species or fungal species,
b) bacterial species comprise Enterococcus faecium, Staphylococcus aureus, Klebsiella pneumoniae, Acinetobacter baumannii, Pseudomonas aeruginosa, Enterobacter species, Escherichia coli, or a combination thereof, and
c) fungal species comprise Aspergillus species, Candida species, Fusarium species, Mucorales (Zygomycetes), Scedosporium species, Curvularia species, Alternaria species, Trichophyton species, Exophiala species, Cladosporium species, Bipolaris species, Penicillium species, Phialophora and Fonsecaea species, or a combination thereof.

14. The device of claim 12, wherein the detectable bacterial species include antibiotic-resistant strains selected from the group consisting of Carbapenem-resistant Acinetobacter baumannii, Methicillin-resistant Staphylococcus aureus (MRSA), Vancomycin-resistant Enterococcus faecium (VRE), Carbapenem-resistant Pseudomonas aeruginosa, and extended-spectrum beta-lactamase-producing bacteria.

15. The device of claim 1, wherein the drainage fluid comprises a body fluid selected from the group consisting of: blood, pus, serous fluid, serosanguineous fluid, seropurulent fluid, lymph, bile, intestinal contents, wound exudate, and combinations thereof.

16. A medical drainage system or analysis setup comprising:
a) means for draining fluid from a patient site or means for holding a collected fluid sample derived therefrom; and
b) the device according to claim 1, operatively associated with said means for draining or holding to analyze said fluid or a headspace associated therewith.

17. The medical drainage system of claim 16, wherein the means for draining fluid comprises a system selected from the group consisting of: Jackson-Pratt, Hemovac, Blake, Redon, Penrose, Vacuum-Assisted Closure (VAC)/Negative Pressure Wound Therapy (NPWT) systems, Capillary drains, Chest Tubes, Pigtail drains, and Silicone drains.

18. A method for monitoring and detecting pathogens associated with a medical drainage system, comprising:
a) providing the device according to claim 1;
b) exposing the sensor array of the device to gaseous analytes present in a headspace associated with drainage fluid from or derived from the medical drainage system;
c) detecting said gaseous analytes using the sensor array;
d) processing data generated by the sensor array using the intelligent analysis module of the device to generate an output, said output comprising at least one of: an indication of pathogen presence, an estimation of microbial load, an assessment aiding in pathogen classification or identification, a differentiation between infectious and non-infectious inflammatory states, or a prediction of infection risk; and
e) communicating or displaying said output using the communication means or the user interface of the device.

19. The method of claim 18, wherein step b) comprises exposing the sensor array to gaseous analytes associated with a drainage fluid sample collected previously from the medical drainage system and analyzed using the device configured as a standalone unit.

20. The method of claim 18, wherein steps c) and d) are performed continuously or periodically over time to monitor dynamic changes indicative of infection progression or resolution.

21. The method of claim 18, further comprising recalibrating the sensor array subsequent to detection and treatment of an infection to enable continued surveillance.

22. The method of claim 18, wherein the detecting and processing occur without requiring prior sample processing or enrichment steps separate from the operation of the medical drainage system and the device when performed in an integrated or in-line configuration.
